(19) United States
(12) Patent Application Publication
(10) Pub. No.: US 2015/0182335 A1
(43) Pub. Date: Jul. 2, 2015

(54) LEFT VENTRICULAR OUTFLOW DEVICE (LVODe)

(71) Applicant: Maxwell Opoku Agyemang, South Miami, FL (US)

(72) Inventor: Maxwell Opoku Agyemang, South Miami, FL (US)

(73) Assignee: PHYSICIANS CREEK INC., South Miami, FL (US)

(21) Appl. No.: 14/145,948

(22) Filed: Jan. 1, 2014

(51) Int. Cl. A61F 2/24

(52) U.S. Cl. CPC A61F 2/2442; A61F 2230/0069

(57) ABSTRACT

Left Ventricular Outflow Device (LVODe) is a medical apparatus implanted to enable normal flow from the left ventricle to the aorta in hypertrophic cardiomyopathy. Hypertrophic cardiomyopathy (HCM) is a congenital disease of the heart with heterogenic genetic phenotypes that result in obstructed flow to the aorta. Echocardiography characterizes HCM as having left ventricular wall thickness greater than 25 mm, asymmetric hypertrophy, left ventricular outflow obstruction, systolic anterior motion of the mitral valve. Patients present with sudden death, perilous arrhythmias, anginal symptoms, and exertional dyspnea. Treatments used for HCM are medical therapy, surgical myectomy and alcohol septal ablation; which are not without adverse effects. In addition, the structural variation of HCM makes no one particular treatment the magic bullet.

Hence the concept of LVODe: a method to modulate the abnormal dynamic flow obstruction to the aorta. The success of the device will hinge on its clinical trials or outcomes.

In Hypertrophic Cardiomyopathy (HCM) there is dynamic outflow obstruction of blood from the left ventricle to the aortic valve during systole. The pressure gradient raised at the outflow tract also creates a suction effect on the anterior cusp of the mitral valve moving it in the flow direction further worsening the left ventricular outflow obstruction. Patients affected by this disease experience sudden cardiac death, perilous ventricular arrhythmias, palpitations, and exercise intolerance. Left Ventricular Outflow Device (LVODe) is essentially tube located juxtaposition to the hypertrophied septum between the interventricular septum and the left atrial-mitral valve structure thereby reducing the gradient, confining the mitral valve apparatus so as to course flow to the aortic valve unobstructed.

### DESCRIPTION OF FIGURES AND VIEWS

FIG. 1: Front page view. Depicted in this figure is 10 which is the hypertrophied interventricular septum obstruction flow from the left ventricle 9 to 1, the aorta. To avert this occurrence, 2 the LVODe is positioned subaortic as a conduit structure and apparatus that will overcome the systolic anterior mitral valve 6 of the 5 the left atrium. In some phenotypes of the disease, the papillary muscles 8 or the chordea tendinea 7 attached to the mitral valve have abnormal morphology creating a primary valvular obstruction to the aorta, and in which case the LVODe 2 will prevent the obstruction. Coursing through the trileaflet cusp of the aortic valve 4 is the spine of the device that projects into spider-like prongs 3 which appose and attach to the walls of the aortic root 11.

FIG. 2: Also on front page view. Conceptually the same drawing as in FIG. 1 with emphasis on the hypertrophied septum and the left ventricular outflow device secured at the root of the aorta.

FIG. 3: Five paramount properties make up the Left Ventricular Outflow Device (LVODe) as seen in this figure. The trumpet-like opening 12 allows inflow from the left ventricle during systole into the stem 2 of the device. The stem is a round tube 20-35 mm in diameter and a projected length of 30-40 mm. Sizes of the device will vary depending on patient factors such as gender, body surface area, and age. The crux of the instrument is to overcome any left ventricular outflow pressure gradient up to and greater than 30 mmHg. The device is curved at the stem and abuts the walls of the left atrium to enable it obliterate the systolic anterior motion of the mitral valve or any presence of abnormal anatomy of the anterior leaflet of the mitral valve. The stem is attached to a spine 14 via a mesh 13 that allows some minimal rotation of the stem during torsional motion of the heart. The spine extends into spider-like prongs 3 that affix to the intima at the root of the aorta. The chain attachment design will prevent the rotational force of the heart from being transmitted to the aortic root. Titanium in its pure or alloy form provides excellent material for manufacturing LVODe, due to its strength, density, corrosive resistance and proven biological compatibility.

FIG. 4: Shows apical long axis view with normal flow from the right ventricle into the left ventricle via the mitral valve in A. B depicts normal septum and valvular apparatus conducting free flow from the outflow tract to the aortic valve.

FIG. 5: Is a variant of HCM connoted by the name 12v H A shows diastolic flow through the mitral valve. B is systolic obstruction as a result of the 12 o'clock abnormal positioning of the papillary muscles and a mitral valve systolic anterior motion v created by hypertrophic septum H. The twin arrows in B represent the mitral valve regurgitation into the left atrium created by the backflow from the obstructed outflow tract. C is the correction with placement of the device 2 reducing the pressure gradient thus allowing flow into the aorta.

FIG. 6: Another phenotypic representation of the disease named 11vH 11 is abnormal position of the papillary muscle at the 11 o'clock position with valvular obstruction v and hypertrophic septum H causing a systolic regurgitation as in B. After implanting LVODe 2 seen in C, the outflow velocity is reduced displacing the mitral anterior leaflet away from the flow volume.

FIG. 7: 10½v H Another abnormal heterogeny of HCM. In this phenotype there is unusual location of the papillary muscle at the 10 o'clock position and in addition abnormal interval and splitting of the papillary muscles 1/2 in combination with a hypertrophied septum H a systolic anterior motion is created v obstructing flow to the aorta shown in B. C has implanted device 2 creating a channel thus obliterating the subaortic stenosis.

FIG. 8: A variant 10vHH of HCM presents as abnormally asymmetric hypertrophy of both lateral wall and septum HH of the left ventricle with abnormal involvement of the mitral valve v causing increased pressure gradient and obstruction to flow in B. LVODe implanted in C improves hemodynamics by allowing continuous flow to the aorta.

FIG. 9: HCM is a disease of wide spectrum commonly obstructing the outflow tract. In the 10vVH variant there is abnormal position of the papillary muscles, abnormal lengthening of the anterior mitral valve leaflet V, which makes the systolic anterior motion v even worse in combination to a hypertrophied septum H as seen in B. In C the curved stem of the Left Ventricular Outflow Device LVODe, conducts flow and asymptotes the leaflet obliterating the obstruction.

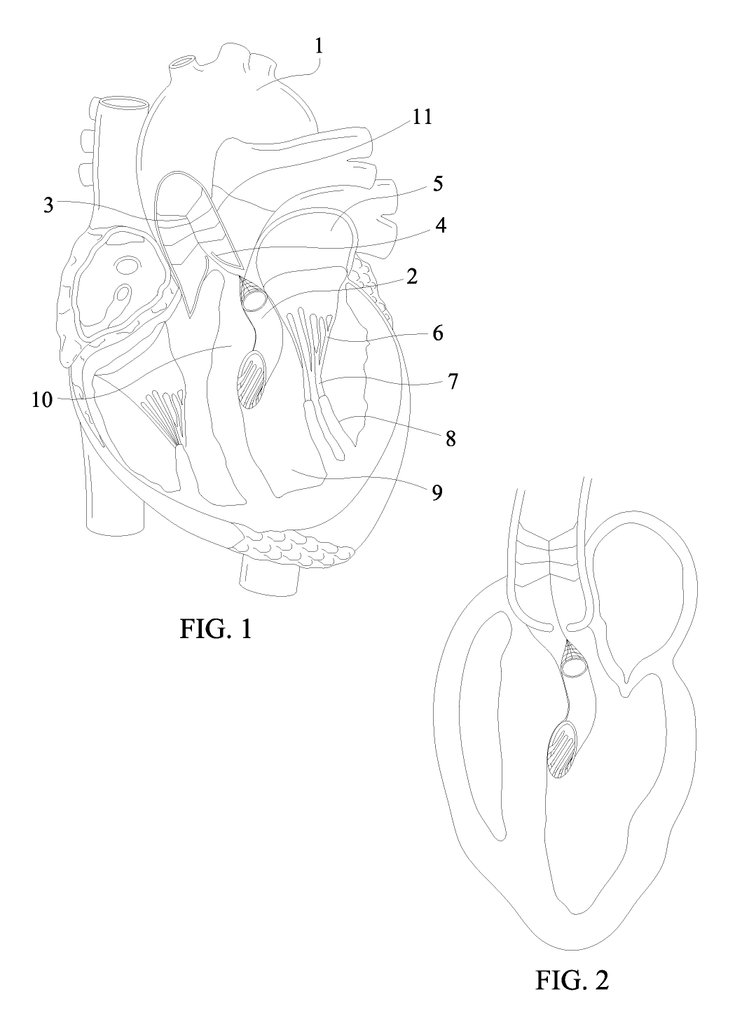

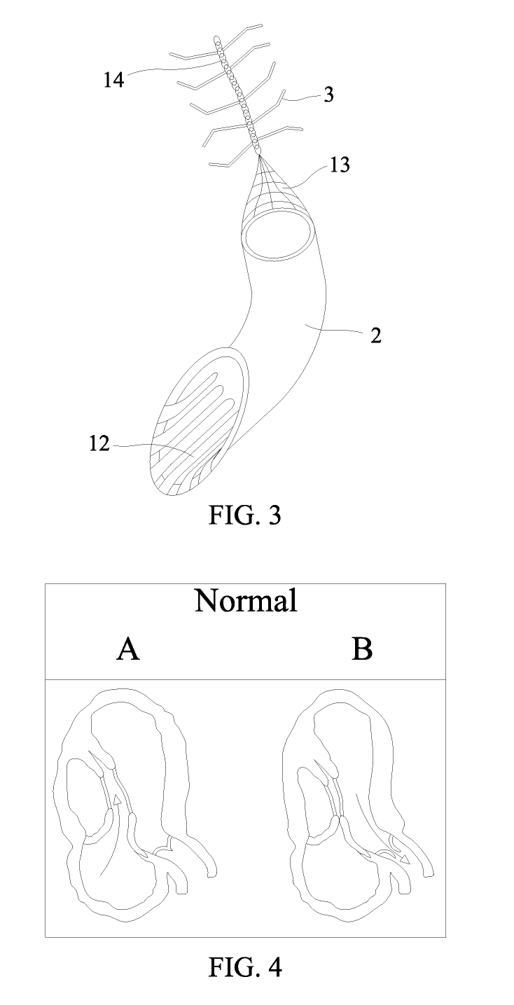

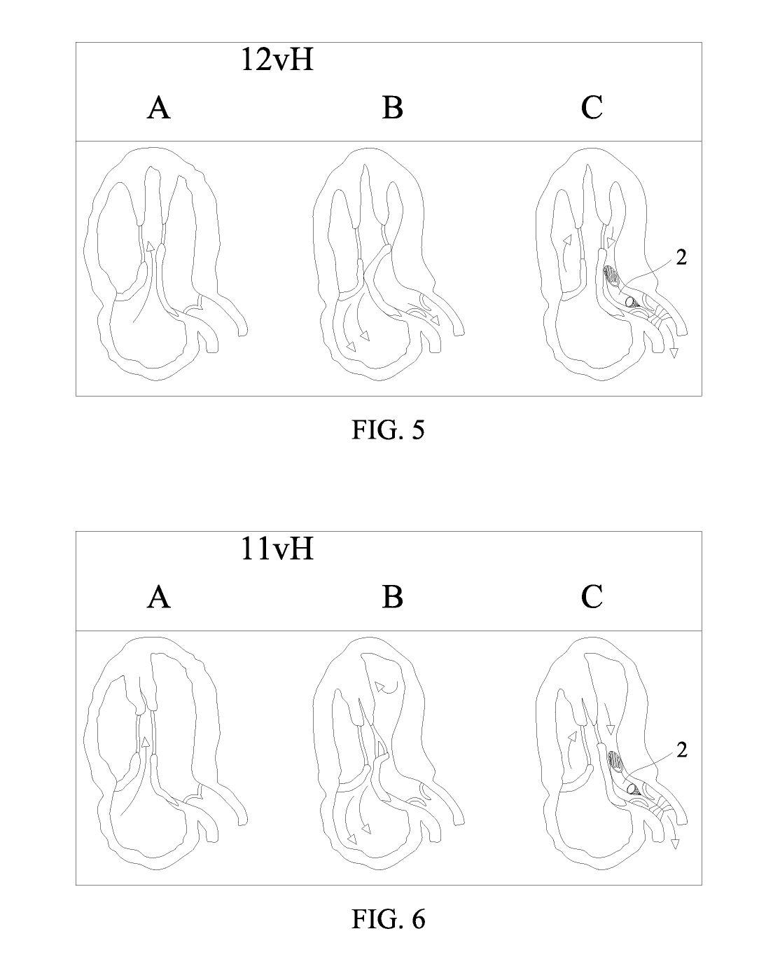

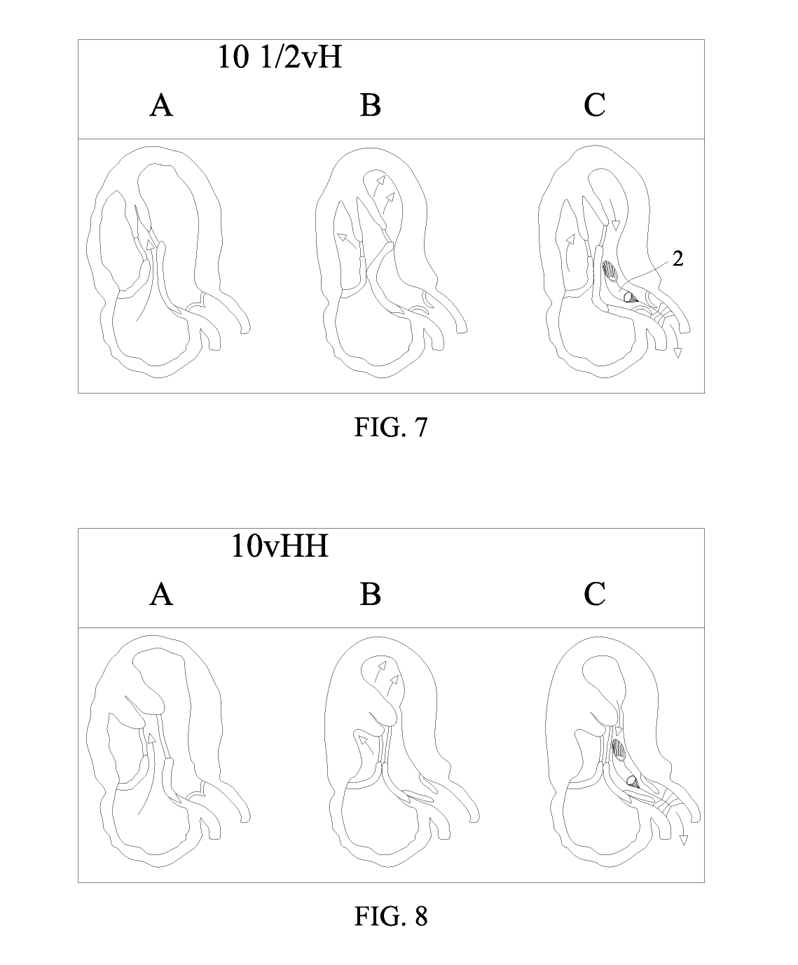

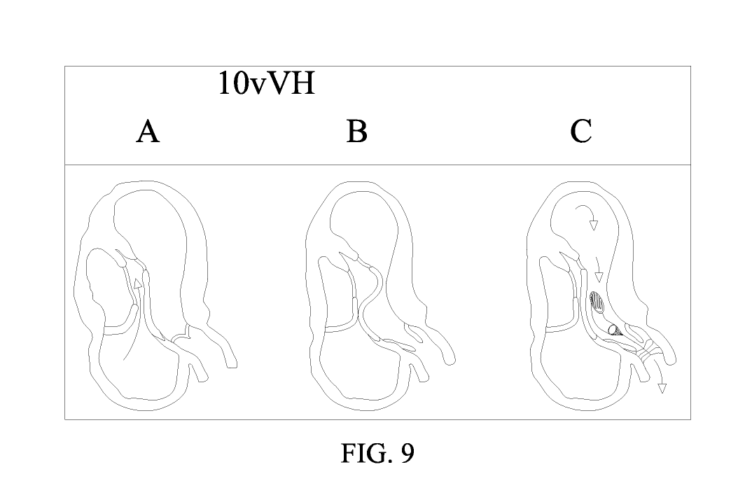

1. Left Ventricular Outflow Device (LVODe) used in hypertrophic obstructive cardiomyopathy to reduce the pressure gradient caused by the outflow tract obstruction.

2. The device in claim 1 equipped with a mesh and a chain system that allows attachment at the aortic root and allows the device to freely move with the torsional motion of the heart.

3. The device in claim 1 wherein the main body is a curved tube and by properties of its size, location and design confines the anterior cusp of the mitral valve thereby obliterating its systolic anterior motion.
